(19) United States
(12) Patent Application Publication
(10) Pub. No.: US 2026/0201242 A1
(43) Pub. Date: Jul. 16, 2026

(54) DIPYRAZOLOPYRIDINE-BASED CORROSION INHIBITOR COMPOSITIONS AND METHODS THEREOF

(71) Applicants: SAUDI ARABIAN OIL COMPANY, Dhahran (SA); King Fahd University of Petroleum and Minerals, Dhahran (SA)

(72) Inventors: Ime BASSEY OBOT, Dhahran (SA); Ahmad A. SOROUR, Dhahran (SA); Tao CHEN, Dhahran (SA); Qiwei WANG, Dhahran (SA); Norah ALJEABAN, Dhahran (SA); Fahd Ibrahim ALGHUNAIMI, Dhahran (SA)

(73) Assignees: SAUDI ARABIAN OIL COMPANY, Dhahran (SA); King Fahd University of Petroleum and Minerals, Dhahran (SA)

(21) Appl. No.: 19/025,425

(22) Filed: Jan. 16, 2025

(51) Int. Cl. C09K 8/54; C07D 471/14; C23F 11/14; E21B 41/02

(52) U.S. Cl. CPC C09K 8/54; C07D 471/14; C23F 11/148; E21B 41/02; C09K 2208/32

(57) ABSTRACT

Methods may comprise providing a corrosion inhibitor composition comprising a dipyrazolopyridine of about 0.1 wt % to about 5 wt % concentration based on a total weight of the corrosion inhibitor composition; a long-chain amine of about 0.1 wt % to about 5 wt % concentration based on the total weight of the corrosion inhibitor composition; a thiourea of about 0.1 wt % to about 5 wt % concentration based on the total weight of the corrosion inhibitor composition; a thiazole of about 0.1 wt % to about 5 wt % concentration based on the total weight of the corrosion inhibitor composition; and a surfactant of about 0.1 wt % to about 5 wt % concentration based on the total weight of the corrosion inhibitor composition; contacting a metal surface with the corrosion inhibitor composition; and allowing the corrosion inhibitor composition to interact with the metal surface so as to inhibit corrosion of the metal surface.

### FIELD OF THE DISCLOSURE

The present disclosure relates generally to corrosion inhibitors and, more particularly, to dipyrazolopyridine-based corrosion inhibitors useful for inhibiting metal corrosion in subterranean formations.

### BACKGROUND OF THE DISCLOSURE

The corrosion of internal pipeline surfaces, particularly at elevated temperatures, is a significant and ongoing challenge in the oil and gas industry. In addition to hydrocarbons, reservoirs frequently produce brine, carbon dioxide, hydrogen sulfide, and organic acids. These substances interact with the metal surfaces of the pipelines, leading to severe degradation and compromising the structural integrity of low-carbon steel pipelines. This persistent issue necessitates advanced strategies for corrosion mitigation.

Corrosion mitigation in oil and gas production environments using chemical inhibitors is particularly challenging under high-temperature conditions (e.g., downhole temperatures greater than 120° C.), in high total dissolved solids (TDS) brines, and in the presence of dissolved carbon dioxide. The extreme temperatures encountered in deep wells and thermal recovery methods pose significant challenges to conventional inhibitor technologies. Traditional organic inhibitors often fail to adequately protect carbon steel assets, leaving the steel vulnerable to localized corrosion in sweet environments. These conditions require inhibitors that are thermally stable and persistent, ensuring uniform filming and effective corrosion protection over extended periods and under harsh conditions.

Recent advancements have led to the development of high-temperature carbon dioxide corrosion inhibitor formulations that incorporate a variety of compounds, including amides, alkynols, mercaptans, piperidines, mercaptopyridines, and suitable solvents for oilfield water systems operating at elevated temperatures. Despite these developments, species such as imidazolines are often favored for their potential efficacy in mitigating corrosion at high temperatures. However, the performance of these inhibitors in the temperature range of 120° C. to 150° C. remains highly system specific, and their effectiveness diminishes significantly at temperatures exceeding 150° C. Additionally, some imidazoline inhibitors may interfere with the formation of protective scales on metal surfaces and may be prone to hydrolysis, limiting their long-term stability and effectiveness.

The high temperatures associated with modern oil extraction techniques, such as deep drilling and enhanced oil recovery methods, necessitate inhibitors that can withstand such harsh environments. Conventional inhibitors often degrade or become ineffective under these conditions, leading to increased maintenance costs, potential failures, and environmental hazards. Consequently, there is a growing need for inhibitors that not only offer high thermal stability but also maintain protective properties in the presence of high TDS brines and carbon dioxide.

### SUMMARY OF THE DISCLOSURE

Various details of the present disclosure are hereinafter summarized to provide a basic understanding. This summary is not an extensive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to an embodiment consistent with the present disclosure, methods for using dipyrazolopyridine-based corrosion inhibitor compositions may comprise: providing a corrosion inhibitor composition comprising: a dipyrazolopyridine having a concentration of about 0.1 wt % to about 5 wt % based on a total weight of the corrosion inhibitor composition; a long-chain amine having a concentration of about 0.1 wt % to about 5 wt % based on the total weight of the corrosion inhibitor composition; a thiourea having a concentration of about 0.1 wt % to about 5 wt % based on the total weight of the corrosion inhibitor composition; a thiazole having a concentration of about 0.1 wt % to about 5 wt % based on the total weight of the corrosion inhibitor composition; and a surfactant having a concentration of about 0.1 wt % to about 5 wt % based on the total weight of the corrosion inhibitor composition; contacting a metal surface with the corrosion inhibitor composition; and allowing the corrosion inhibitor composition to interact with the metal surface so as to inhibit corrosion of the metal surface.

In another embodiment, methods for preparing dipyrazolopyridine-based corrosion inhibitor compositions may comprise: providing a hydrazine and an acetoacetate; interacting the hydrazine with the acetoacetate at a set of reaction conditions to form a product; and interacting the product with an aldehyde and an ammonium acetate to form a dipyrazolopyridine.

In a further embodiment, dipyrazolopyridine-based corrosion inhibitor compositions may comprise: a dipyrazolopyridine having a concentration of about 0.1 wt % to about 5 wt % based on a total weight of the composition; a long-chain amine having a concentration of about 0.1 wt % to about 5 wt % based on the total weight of the composition; a thiourea having a concentration of about 0.1 wt % to about 5 wt % based on the total weight of the composition; a thiazole having a concentration of about 0.1 wt % to about 5 wt % based on the total weight of the composition; and a surfactant having a concentration of about 0.1 wt % to about 5 wt % based on the total weight of the composition

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

The FIGURE is a graph of the corrosion rate of a carbon steel over time when exposed to a corrosion inhibitor composition of the present disclosure or a brine.

### DETAILED DESCRIPTION

Embodiments in accordance with the present disclosure generally relate to corrosion inhibitors and, more particularly, to dipyrazolopyridine-based corrosion inhibitors useful for inhibiting metal corrosion in subterranean formations. As mentioned previously, there is an increasing demand for inhibitors that provide high thermal stability while also retaining their protective properties in high TDS brines and carbon dioxide environments. The present disclosure addresses the foregoing with dipyrazolopyridine-based corrosion inhibitor compositions and methods thereof.

Methods for corrosion inhibition may comprise providing a corrosion inhibitor composition comprising a dipyrazolopyridine having a concentration of about 0.1 wt % to about 5 wt % based on a total weight of the corrosion inhibitor composition; a long-chain amine having a concentration of about 0.1 wt % to about 5 wt % based on the total weight of the corrosion inhibitor composition; a thiourea having a concentration of about 0.1 wt % to about 5 wt % based on the total weight of the corrosion inhibitor composition; a thiazole having a concentration of about 0.1 wt % to about 5 wt % based on the total weight of the corrosion inhibitor composition; and a surfactant having a concentration of about 0.1 wt % to about 5 wt % based on the total weight of the corrosion inhibitor composition; contacting a metal surface with the corrosion inhibitor composition; and allowing the corrosion inhibitor composition to interact with the metal surface so as to inhibit corrosion of the metal surface.

In another embodiment, methods for preparing dipyrazolopyridine-based corrosion inhibitors may comprise providing a hydrazine and an acetoacetate; interacting the hydrazine with the acetoacetate at a set of reaction conditions to form a product; and interacting the product with an aldehyde and an ammonium acetate to form a dipyrazolopyridine.

Furthermore, dipyrazolopyridine-based corrosion inhibitor compositions may comprise a dipyrazolopyridine having a concentration of about 0.1 wt % to about 5 wt % based on a total weight of the composition; a long-chain amine having a concentration of about 0.1 wt % to about 5 wt % based on the total weight of the composition; a thiourea having a concentration of about 0.1 wt % to about 5 wt % based on the total weight of the composition; a thiazole having a concentration of about 0.1 wt % to about 5 wt % based on the total weight of the composition; and a surfactant having a concentration of about 0.1 wt % to about 5 wt % based on the total weight of the composition.

The dipyrazolopyridine component of the corrosion inhibitors suitable for the present disclosure may comprise Formula (I).

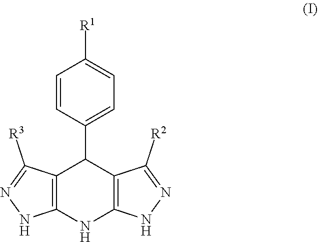

In Formula (I), R¹ may be a methoxy, a carboxylate, an aromatic amide, a thiol, a nitrile, an azo, a pyridyl, a bromophenyl, or a sulfonic acid. R² may be an alkyl, a mercapto, a carboxylate, an aromatic carbonyl, a hydroxy, a methyl amino, an amide, or an ester. R³ may be an alkyl, an amide, an amino, a mercapto, a hydroxy, an ester, or a carboxylic acid.

The dipyrazolopyridine may be produced using any suitable method. For example, the dipyrazolopyridine may be produced by providing a hydrazine (e.g., a hydrazine hydrate) and an acetoacetate (e.g., an ethyl acetoacetate) and interacting the hydrazine and the acetoacetate at a set of reaction conditions to form a product. The set of reaction conditions may, for example, comprise agitating the hydrazine and the acetoacetate (e.g., by sonication) for about 1 minute to about 30 minutes (or about 1 minute to about 20 minutes, or about 1 minute to about 10 minutes, or about 1 minute to about 5 minutes, or about 5 minutes to about 30 minutes, or about 5 minutes to about 20 minutes, or about 5 minutes to about 10 minutes, or about 10 minutes to about 30 minutes, or about 10 minutes to about 20 minutes, or about 20 minutes to about 30 minutes) at a temperature of about 20° C. to about 30° C. (or about 20° C. to about 25° C., or about 25° C. to about 30° C.), such as room temperature. The product may further interact with an aldehyde and an ammonium acetate to form the dipyrazolopyridine.

The dipyrazolopyridine may, for example, have a concentration of about 0.1 wt % to about 5 wt % (or about 0.1 wt % to about 4 wt %, or about 0.1 wt % to about 3 wt %, or about 0.1 wt % to about 2.5 wt %, or about 0.5 wt % to about 5 wt %, or about 0.5 wt % to about 4 wt %, or about 0.5 wt % to about 3 wt %, or about 0.5 wt % to about 2.5 wt %, or about 1 wt % to about 5 wt %, or about 1 wt % to about 4 wt %, or about 1 wt % to about 3 wt %, or about 1 wt % to about 2.5 wt %, or about 2.5 wt % to about 5 wt %, or about 2.5 wt % to about 4 wt %, or about 2.5 wt % to about 3 wt %), based on a total weight of the corrosion inhibitor composition.

The long-chain amine component of the corrosion inhibitors suitable for the present disclosure may comprise a hydrocarbyl chain having 1 to 18 carbons. For example, the long-chain amine may comprise Formula (II).

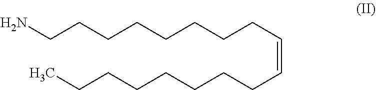 [SMILES: CCCCCCCC/C=C\CCCCCCCCN]

The long-chain amine may further have a concentration of about 0.1 wt % to about 5 wt % (or about 0.1 wt % to about 4 wt %, or about 0.1 wt % to about 3 wt %, or about 0.1 wt % to about 1.25 wt %, or about 0.5 wt % to about 5 wt %, or about 0.5 wt % to about 4 wt %, or about 0.5 wt % to about 3 wt %, or about 0.5 wt % to about 1.25 wt %, or about 1 wt % to about 5 wt %, or about 1 wt % to about 4 wt %, or about 1 wt % to about 3 wt %, or about 1 wt % to about 1.25 wt %, or about 1.25 wt % to about 5 wt %, or about 1.25 wt % to about 4 wt %, or about 1.25 wt % to about 3 wt %) based on the total weight of the corrosion inhibitor composition.

The thiourea component of the corrosion inhibitors suitable for the present disclosure may comprise a diphenyl thiourea. For example, the thiourea may comprise Formula (III).

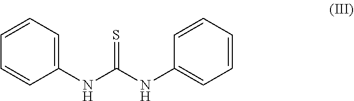 [SMILES: S=C(Nc1ccccc1)Nc1ccccc1]

The thiourea may further have a concentration of about 0.1 wt % to about 5 wt % (or about 0.1 wt % to about 4 wt %, or about 0.1 wt % to about 3 wt %, or about 0.1 wt % to about 1.25 wt %, or about 0.5 wt % to about 5 wt %, or about 0.5 wt % to about 4 wt %, or about 0.5 wt % to about 3 wt %, or about 0.5 wt % to about 1.25 wt %, or about 1 wt % to about 5 wt %, or about 1 wt % to about 4 wt %, or about 1 wt % to about 3 wt %, or about 1 wt % to about 1.25 wt %, or about 1.25 wt % to about 5 wt %, or about 1.25 wt % to about 4 wt %, or about 1.25 wt % to about 3 wt %) based on the total weight of the corrosion inhibitor composition.

The thiazole component of the corrosion inhibitors suitable for the present disclosure may comprise a benzothiazole. For example, the thiazole may comprise Formula (IV).

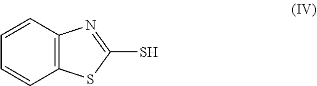

The thiazole may further have a concentration of about 0.1 wt % to about 5 wt % (or about 0.1 wt % to about 4 wt %, or about 0.1 wt % to about 3 wt %, or about 0.1 wt % to about 1.25 wt %, or about 0.5 wt % to about 5 wt %, or about 0.5 wt % to about 4 wt %, or about 0.5 wt % to about 3 wt %, or about 0.5 wt % to about 1.25 wt %, or about 1 wt % to about 5 wt %, or about 1 wt % to about 4 wt %, or about 1 wt % to about 3 wt %, or about 1 wt % to about 1.25 wt %, or about 1.25 wt % to about 5 wt %, or about 1.25 wt % to about 4 wt %, or about 1.25 wt % to about 3 wt %) based on the total weight of the corrosion inhibitor composition.

The surfactant component of the corrosion inhibitors suitable for the present disclosure may comprise a non-ionic surfactant (e.g., a polysorbate). For example, the surfactant may comprise Formula (V)

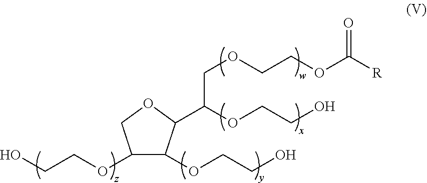

In Formula (V), variables w, x, y, and z represent integers of 1 to 100. The surfactant may further have a concentration of about 0.1 wt % to about 5 wt % (or about 0.1 wt % to about 4 wt %, or about 0.1 wt % to about 3 wt %, or about 0.1 wt % to about 1.25 wt %, or about 0.5 wt % to about 5 wt %, or about 0.5 wt % to about 4 wt %, or about 0.5 wt % to about 3 wt %, or about 0.5 wt % to about 1.25 wt %, or about 1 wt % to about 5 wt %, or about 1 wt % to about 4 wt %, or about 1 wt % to about 3 wt %, or about 1 wt % to about 1.25 wt %, or about 1.25 wt % to about 5 wt %, or about 1.25 wt % to about 4 wt %, or about 1.25 wt % to about 3 wt %) based on the total weight of the corrosion inhibitor composition.

A remainder of the corrosion inhibitor composition may comprise a solvent. Any suitable solvent may be used, including, but not limited to, glycol, dimethyl sulfoxide (DMSO), the like, and any combination thereof. The surfactant may be present at a concentration of about 45 wt % to about 99 wt %.

As a component of a treatment fluid, the corrosion inhibitor composition may be present at concentrations of about 10 ppm to about 10,000 ppm (or about 10 ppm to about 1,000 ppm, or about 10 ppm to about 100 ppm, or about 100 ppm to about 10,000 ppm, or about 100 ppm to about 1,000 ppm, or about 1,000 ppm to about 10,000 ppm), based on the total weight of the treatment fluid. The treatment fluid may further comprise any other suitable component that may be a diluent of the corrosion inhibitor composition. For example, the treatment fluid may comprise formation water, produced water, brine, sea water, groundwater, potable and/or non-potable water, or any combination thereof.

The corrosion inhibitor compositions of the present disclosure may be used in subterranean environments that have a bottom hole temperature ranging from about 70° F. to about 500° F. (or about 70° F. to about 300° F., or about 200° F. to about 300° F., or about 300° F. to about 500° F.).

The corrosion inhibitor compositions of the present disclosure may optionally include one or more of a variety of well-known additives, such as gel stabilizers, salts, fluid loss control additives, scale inhibitors, catalysts, clay stabilizers, biocides, bactericides, friction reducers, gases, foaming agents, iron control agents, solubilizers, pH adjusting agents (e.g., buffers), the like, and any combination thereof. Those of ordinary skill in the art, with the benefit of this disclosure, may be able to determine the appropriate additives for a particular application.

The metal surfaces to be protected by the corrosion inhibitor compositions of the present disclosure may include any metal surface susceptible to corrosion in an acidic environment (e.g., due to dissolved carbon dioxide) including, but not limited to, ferrous metals, low alloy metals (e.g., N-80 Grade), stainless steel (e.g., 13 Cr), copper alloys, brass, nickel alloys, and duplex stainless steel alloys. Such metal surfaces include downhole piping, downhole tools, and the like.

The present disclosure is further directed to the following non-limiting clauses:

Clause 1. A method comprising:
providing a corrosion inhibitor composition comprising:
   a dipyrazolopyridine having a concentration of about 0.1 wt % to about 5 wt % based on a total weight of the corrosion inhibitor composition;
   a long-chain amine having a concentration of about 0.1 wt % to about 5 wt % based on the total weight of the corrosion inhibitor composition;
   a thiourea having a concentration of about 0.1 wt % to about 5 wt % based on the total weight of the corrosion inhibitor composition;
   a thiazole having a concentration of about 0.1 wt % to about 5 wt % based on the total weight of the corrosion inhibitor composition; and
   a surfactant having a concentration of about 0.1 wt % to about 5 wt % based on the total weight of the corrosion inhibitor composition;
contacting a metal surface with the corrosion inhibitor composition; and
allowing the corrosion inhibitor composition to interact with the metal surface so as to inhibit corrosion of the metal surface.

Clause 2. The method of clause 1, wherein the dipyrazolopyridine comprises Formula (I);

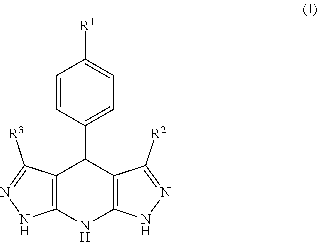

wherein:
R¹ is methoxy, carboxylate, aromatic amide, thiol, nitrile, azo, pyridyl, bromophenyl, or sulfonic acid;
R² is alkyl, mercapto, carboxylate, aromatic carbonyl, hydroxy, methyl amino, amide, or ester; and
R³ is alkyl, amide, amino, mercapto, hydroxy, ester, or carboxylic acid.

Clause 3. The method of clause 1 or clause 2, wherein the long-chain amine comprises a hydrocarbyl chain having 1 to 18 carbons.

Clause 4. The method of any one of clauses 1-3, wherein the thiourea comprises a diphenyl thiourea.

Clause 5. The method of any one of clauses 1-4, wherein the thiazole comprises a benzothiazole.

Clause 6. The method of any one of clauses 1-5, wherein the surfactant comprises a non-ionic surfactant.

Clause 7. The method of any one of clauses 1-6, wherein the non-ionic surfactant comprises a polysorbate.

Clause 8. The method of any one of clauses 1-7, wherein the corrosion inhibitor further comprises a solvent, the solvent comprising glycol, dimethyl sulfoxide, or a combination thereof.

Clause 9. A method comprising:
providing a hydrazine and an acetoacetate;
interacting the hydrazine with the acetoacetate at a set of reaction conditions to form a product; and
interacting the product with an aldehyde and an ammonium acetate to form a dipyrazolopyridine.

Clause 10. The method of clause 9, wherein the dipyrazolopyridine comprises Formula (I);

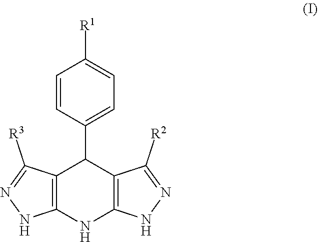

wherein:
R¹ is methoxy, carboxylate, aromatic amide, thiol, nitrile, azo, pyridyl, bromophenyl, or sulfonic acid;
R² is alkyl, mercapto, carboxylate, aromatic carbonyl, hydroxy, methyl amino, amide, or ester; and
R³ is alkyl, amide, amino, mercapto, hydroxy, ester, or carboxylic acid.

Clause 11. The method of clause 9 or clause 10, wherein the hydrazine comprises a hydrazine hydrate.

Clause 12. The method of any one of clauses 9-11, wherein the acetoacetate comprises an ethyl acetoacetate.

Clause 13. The method of any of clauses 9-12, wherein the set of reaction conditions comprises sonicating the hydrazine and the acetoacetate for about 1 minute to about 30 minutes at a temperature of about 20° C. to about 30° C.

Clause 14. A composition comprising:
a dipyrazolopyridine having a concentration of about 0.1 wt % to about 5 wt % based on a total weight of the composition;
a long-chain amine having a concentration of about 0.1 wt % to about 5 wt % based on the total weight of the composition;
a thiourea having a concentration of about 0.1 wt % to about 5 wt % based on the total weight of the composition;
a thiazole having a concentration of about 0.1 wt % to about 5 wt % based on the total weight of the composition; and
   a surfactant having a concentration of about 0.1 wt % to about 5 wt % based on the total weight of the composition.

Clause 15. The composition of clause 14, wherein the dipyrazolopyridine comprises Formula (I);

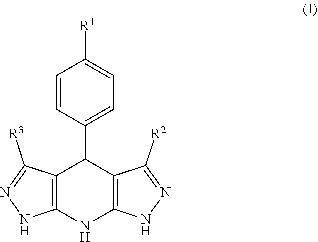

wherein:
R¹ is methoxy, carboxylate, aromatic amide, thiol, nitrile, azo, pyridyl, bromophenyl, or sulfonic acid;
R² is alkyl, mercapto, carboxylate, aromatic carbonyl, hydroxy, methyl amino, amide, or ester; and
R³ is alkyl, amide, amino, mercapto, hydroxy, ester, or carboxylic acid.

Clause 16. The composition of clause 14 or clause 15, wherein the long-chain amine comprises a hydrocarbyl chain having 1 to 18 carbons.

Clause 17. The composition of any one of clauses 14-16, wherein the thiourea comprises a diphenyl thiourea.

Clause 18. The composition of any one of clauses 14-17, wherein the thiazole comprises a benzothiazole.

Clause 19. The composition of any one of clauses 14-18, wherein the surfactant comprises a polysorbate.

Clause 20. The composition of any one of clauses 14-19, further comprising a solvent, the solvent comprising glycol, dimethyl sulfoxide, or a combination thereof.

### EXAMPLES

A corrosion inhibitor composition consistent with the present disclosure was prepared according to Table 1.

TABLE 1

| Component | Concentration (wt %) |
|---|---|
| Pyrazolopyridine | 2.5 |
| Benzothiazole | 1.25 |
| Diphenyl thiourea | 1.25 |
| Polysorbate 80 | 1.25 |
| Alkylamine | 1.25 |
| Glycol | 45 |
| Dimethyl sulfoxide (DMSO) | 47.5 |

Corrosion evaluation was conducted using cylindrical carbon steel C1018, supplied by GAMRY®, USA. The samples were prepared by grinding with silicon carbide paper of grit sizes #400, #600, and #800, followed by thorough washing with distilled water, degreasing with acetone, and air drying. These prepared samples were immediately used for corrosion testing.

A standard 1000 L corrosion cell was employed, utilizing the Gamry Reference 1010E Potentiostat/Galvanostat and Echem Analyst software suite for electrochemical measurements. The experimental setup included five ports: a cylindrical C1018 carbon steel as the working electrode, a saturated calomel electrode (SCE) as the reference electrode, a cylindrical graphite rod as the counter electrode, a CO₂ gas sparger, and a port for a pH and temperature meter.

The corrosion inhibitor composition and a control composition consisting of only synthetic formation brine (Table 2) were tested according to ASTM standards G59 and G3 using carbon steel coupons (C1018) at a temperature of 70° C.

TABLE 2

| Brine Component | Concentration (g/L) |
|---|---|
| NaCl | 46.7 |
| CaCl₂•2H₂O | 70.4 |
| KCl | 6.6 |
| MgCl₂•6H₂O | 4.38 |
| SrCl₂•6H₂O | 3.95 |
| BaSO₄ | 0.0019 |

CO₂ gas was bubbled into the brine to de-aerate the solution for the first two hours and continuously bubbled throughout the experiment to simulate sweet corrosive conditions. The solution was stirred at 500 rpm using a magnetic stirrer. After 2 hours of CO₂ purging, the system was monitored for 1 hour to ensure a stable open circuit potential (OCP). Linear polarization resistance (LPR) measurements were then initiated, recording the corrosion rate every 10 minutes. A pre-corrosion period of 2 hours was allowed before injecting the corrosion inhibitor formulation at a concentration of 10 ppm. The test duration was 24 hours to allow the corrosion rate to equilibrate. The performance of the corrosion inhibitor was calculated as percent corrosion protection using the pre-corrosion rate as the uninhibited baseline (Equation 1).

$$\% Corrosion Protection ( Eff ) = \frac{{CR}_{pc} - {CR}_{ih}}{{CR}_{pc}} \times 100 \% \qquad Equation 1$$

In Equation 1, CR_{pc} is the corrosion rate before injection of the inhibitor and CRᵢₕ is the stabilized corrosion rate after the addition of the corrosion inhibitor.

The autoclave test evaluated the performance of the corrosion inhibitor composition under high-temperature, high-pressure, and high-shear stress conditions. A pre-weighed carbon steel coupon was suspended in a 2.5 L solution of formation water brine (Table 2). The brine was de-aerated with nitrogen gas for 3 hours. The autoclave was then sealed, de-aerated with nitrogen gas for another hour, pressurized to 100 psi with CO₂, and heated to 120° C. A flow speed of 500 rpm was maintained, and the coupons were exposed for 24 hours. An inhibitor concentration of 500 ppm was used. After 24 hours, the coupons were cleaned with Super Clark's solution (5 g/L N,N-dibutylthiourea dissolved in 18% HCl), reweighed, and the corrosion rate was calculated according to ASTM standard G1 (Equation 2).

$$Corrosion rate = \frac{87600 \times W}{A \times T \times D}$$

In Equation 2, W is the weight lost (g), A is the surface area (cm²), T is the time of exposure (hr), and D is the density (7.86 g/cm³ for carbon steel).

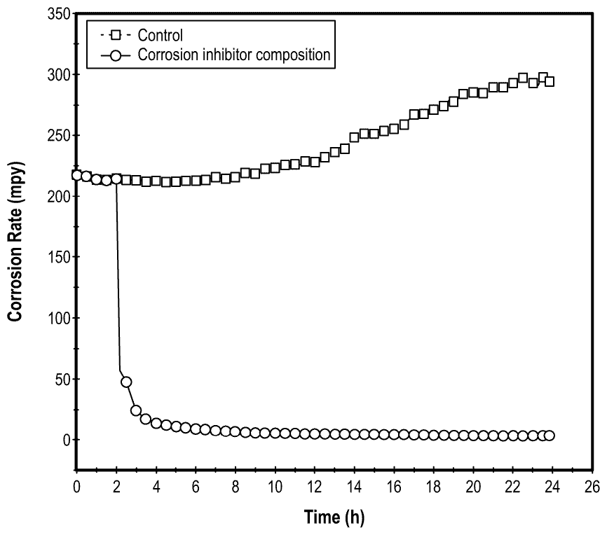

The effectiveness of the corrosion inhibitor composition was evaluated for C1018 steel at 70° C. after a 2-hour pre-corrosion period using a synthetic brine solution (Table 2). The evaluation was conducted with 10 ppm of the corrosion inhibitor. The LPR curves (FIG. 1) show that the corrosion rate without the inhibitor reached 300 mpy. The addition of 10 ppm of NAM 9 reduced the corrosion rate from 214.4 mpy to 3.28 mpy, achieving an inhibition efficiency of 98.47%.

The HTHP autoclave test demonstrated the corrosion rate of the control was 250.6 mpy, while the inhibited sample with the corrosion inhibitor composition showed a corrosion rate of 30.3 mpy, resulting in an efficiency of 87.9% under severe conditions (120° C., 100 psi, 500 rpm). Visual inspection of the carbon steel revealed significant corrosion in the control solution and good performance in the corrosion inhibitor composition.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains,” “containing,” “includes,” “including,” “comprises,” and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

While the present disclosure has been described with respect to a limited number of embodiments, those skilled in the art, having benefit of this disclosure, will appreciate that other embodiments may be devised which do not depart from the scope of the disclosure as described herein. Accordingly, the scope of the disclosure should be limited only by the attached claims.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by one or more embodiments described herein. At the very least, and not as an attempt to limit the application of the doctrine of equivalents to the scope of the claim, each numerical parameter should at least be construed in light of the number of reported significant digits and by applying ordinary rounding techniques.

The invention claimed is:

1. A method comprising:
providing a corrosion inhibitor composition comprising:
a dipyrazolopyridine having a concentration of about 0.1 wt % to about 5 wt % based on a total weight of the corrosion inhibitor composition;
a long-chain amine having a concentration of about 0.1 wt % to about 5 wt % based on the total weight of the corrosion inhibitor composition;
a thiourea having a concentration of about 0.1 wt % to about 5 wt % based on the total weight of the corrosion inhibitor composition;
a thiazole having a concentration of about 0.1 wt % to about 5 wt % based on the total weight of the corrosion inhibitor composition; and
a surfactant having a concentration of about 0.1 wt % to about 5 wt % based on the total weight of the corrosion inhibitor composition;
contacting a metal surface with the corrosion inhibitor composition; and
allowing the corrosion inhibitor composition to interact with the metal surface so as to inhibit corrosion of the metal surface.

2. The method of claim 1, wherein the dipyrazolopyridine comprises Formula (I); 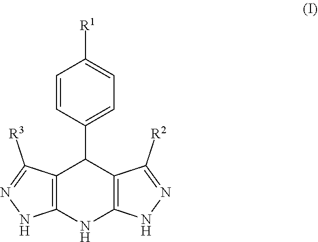
wherein:
R¹ is methoxy, carboxylate, aromatic amide, thiol, nitrile, azo, pyridyl, bromophenyl, or sulfonic acid;
R² is alkyl, mercapto, carboxylate, aromatic carbonyl, hydroxy, methyl amino, amide, or ester; and
R³ is alkyl, amide, amino, mercapto, hydroxy, ester, or carboxylic acid.

3. The method of claim 1, wherein the long-chain amine comprises a hydrocarbyl chain having 1 to 18 carbons.

4. The method of claim 1, wherein the thiourea comprises a diphenyl thiourea.

5. The method of claim 1, wherein the thiazole comprises a benzothiazole.

6. The method of claim 1, wherein the surfactant comprises a non-ionic surfactant.

7. The method of claim 6, wherein the non-ionic surfactant comprises a polysorbate.

8. The method of claim 1, wherein the corrosion inhibitor further comprises a solvent, the solvent comprising glycol, dimethyl sulfoxide, or a combination thereof.

9. A method comprising:
providing a hydrazine and an acetoacetate;
interacting the hydrazine with the acetoacetate at a set of reaction conditions to form a product; and
interacting the product with an aldehyde and an ammonium acetate to form a dipyrazolopyridine.

10. The method of claim 9, wherein the dipyrazolopyridine comprises Formula (I); 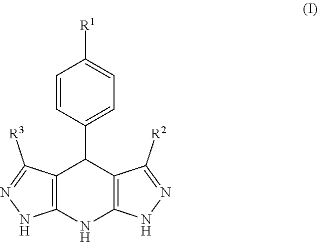
wherein:
R¹ is methoxy, carboxylate, aromatic amide, thiol, nitrile, azo, pyridyl, bromophenyl, or sulfonic acid;
R² is alkyl, mercapto, carboxylate, aromatic carbonyl, hydroxy, methyl amino, amide, or ester; and
R³ is alkyl, amide, amino, mercapto, hydroxy, ester, or carboxylic acid.

11. The method of claim 9, wherein the hydrazine comprises a hydrazine hydrate.

12. The method of claim 9, wherein the acetoacetate comprises an ethyl acetoacetate.
